(19) United States
(12) Patent Application Publication
(10) Pub. No.: US 2009/0171269 A1
(43) Pub. Date: Jul. 2, 2009

(54) Infusion Device and Methods Therefor

(75) Inventors: R. Curtis Jennewine, San Francisco, CA (US); Denyse M. Collins, Derry, NH (US)

Correspondence Address: JACKSON & CO., LLP, 6114 LA SALLE AVENUE, #507, OAKLAND, CA, 94611-2802, US

(73) Assignee: Abbott Diabetes Care, Inc., Alameda, CA (US)

(21) Appl. No.: 11/427,587

(22) Filed: Jun. 29, 2006

(51) Int. Cl. A61M 5/168

(52) U.S. Cl. 604/67

(57) ABSTRACT

Method and apparatus for providing retractable infusion tubing for an infusion device and a jog wheel user interface for an analyte monitoring system receiver and/or an infusion device control unit is provided.

### BACKGROUND

With increasing use of pump therapy for Type 1 diabetic patients, young and old alike, the importance of controlling the infusion device such as external infusion pumps is evident. Indeed, presently available external infusion devices typically include an input mechanism such as buttons through which the patient may program and control the infusion device. Such infusion devices also typically include a user interface such as a display which is configured to display information relevant to the patient's infusion progress, status of the various components of the infusion device, as well as other programmable information such as patient specific basal profiles.

The external infusion devices are typically connected to an infusion set which includes a cannula that is placed transcutaneously through the skin of the patient to infuse a select dosage of insulin based on the infusion device's programmed basal rates or any other infusion rates as prescribed by the patient's doctor. Generally, the patient is able to control the pump to administer additional doses of insulin during the course of wearing and operating the infusion device such as for, administering a carbohydrate bolus prior to a meal. Certain infusion devices include food database that has associated therewith, an amount of carbohydrate, so that the patient may better estimate the level of insulin dosage needed for, for example, calculating a bolus amount.

Programming and controlling the pump functions are typically performed by the patient using the pump user interface which includes input buttons and a display. Typically, depending on the type of the infusion device, the amount of information which is provided to the user generally focus on infusion management such as programming temporary basals, bolus calculation, and the like, in addition to the device operational functions such as alerts for occlusion detection. Given the decreasing cost of microprocessors, and increasing sophistication of patients and users of infusion devices, it would be desirable to provide additional features and functionalities to improve user interface capabilities of such devices.

Indeed, it would be desirable to have an approach to provide user interface features which provide easy of use and robust functionalities in analyte monitoring and therapy management systems.

### SUMMARY OF THE INVENTION

In accordance with the various embodiments of the present invention, there are provided methods and system for an infusion device with improved user interface unit, and a retractable infusion tubing mechanism, and other features for providing ease of use and improved functionality of the infusion device.

These and other objects, features and advantages of the present invention will become more fully apparent from the following detailed description of the embodiments, the appended claims and the accompanying drawings.

### BRIEF DESCRIPTION OF THE DRAWINGS

FIG. 1 is a block diagram illustrating a therapy management system for practicing one embodiment of the present invention;

FIG. 2 is a block diagram of an fluid delivery device of FIG. 1 in one embodiment of the present invention;

FIG. 3 is a block diagram of an fluid delivery device with a jog wheel user interface and retractable tubing in one embodiment of the present invention;

FIG. 4 is a detailed view of the retractable infusion tubing unit of the infusion device of FIG. 3 in one embodiment of the present invention;

FIG. 5 is a detailed view of the retractable infusion tubing core segment of the retractable infusion tubing section of FIG. 4 in one embodiment of the present invention; and

FIGS. 6A and 6B are top planar view and side cross-sectional view, respectively, of the retractable infusion tubing unit in accordance with another embodiment of the present invention.

### DETAILED DESCRIPTION

As described below, within the scope of the present invention, there are provided a retractable infusion tubing mechanism integrated with an external infusion device to provide adjustments to the infusion tubing length and thus providing additional comfort and ease of wear to the user. In addition, there is provided a simple easy to use jog wheel provided on the housing of the external infusion device for providing user input commands to the infusion device.

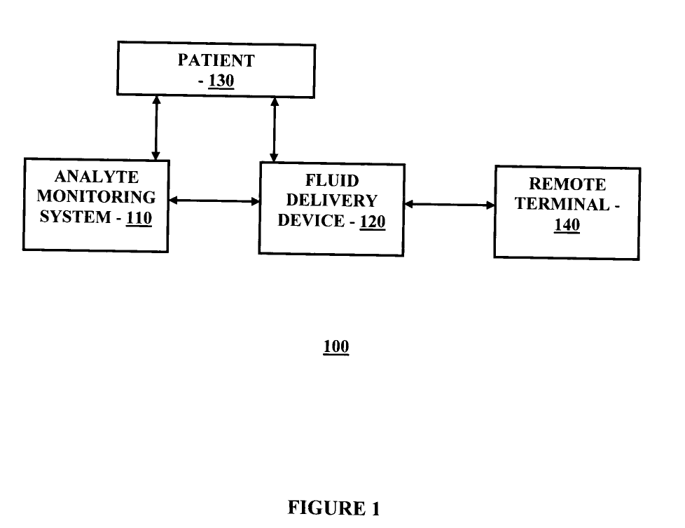

FIG. 1 is a block diagram illustrating a therapy management system for practicing one embodiment of the present invention. Referring to FIG. 1, the therapy management system 100 includes an analyte monitoring system 110 operatively coupled to an fluid delivery device 120, which may be in turn, operatively coupled to a remote terminal 140. As shown the Figure, the analyte monitoring system 110 is, in one embodiment, coupled to the patient 130 so as to monitor or measure the analyte levels of the patient. Moreover, the fluid delivery device 120 is coupled to the patient using, for example, and infusion set and tubing connected to a cannula (not shown) that is placed transcutaneously through the skin of the patient so as to infuse medication such as, for example, insulin, to the patient.

Referring to FIG. 1, in one embodiment the analyte monitoring system 110 in one embodiment may include one or more analyte sensors subcutaneously positioned such that at least a portion of the analyte sensors are maintained in fluid contact with the patient's analytes. The analyte sensors may include, but not limited to short term subcutaneous analyte sensors or transdermal analyte sensors, for example, which are configured to detect analyte levels of a patient over a predetermined time period, and after which, a replacement of the sensors is necessary.

The analyte monitoring system 110 includes one or more analyte sensors that is coupled to a respective one or more of a data transmitter unit which is configured to receive one or more signals from the respective analyte sensors corresponding to the detected analyte levels of the patient, and to transmit the information corresponding to the detected analyte levels to a receiver device, and/or fluid delivery device 120. That is, over a communication link, the transmitter units may be configured to transmit data associated with the detected analyte levels periodically, and/or intermittently and repeatedly to one or more other devices such as the fluid delivery device and/or the remote terminal 140 for further data processing and analysis.

In one aspect, each of the one or more receiver device of the analyte monitoring system 110 and the fluid delivery device includes a user interface unit which may include a display unit and/or an audio output unit such as, for example, a speaker, and/or any other suitable user interface mechanism for displaying or informing the user of such devices.

The transmitter units of the analyte monitoring system 110 may in one embodiment be configured to transmit the analyte related data substantially in real time to the fluid delivery device 120 and/or the remote terminal 140 after receiving it from the corresponding analyte sensors such that the analyte level such as glucose level of the patient 130 may be monitored in real time. In one aspect, the analyte levels of the patient may be obtained using one or more of a discrete blood glucose testing devices such as blood glucose meters, or a continuous analyte monitoring systems such as continuous glucose monitoring systems.

Additional analytes that may be monitored, determined or detected the analyte monitoring system 110 include, for example, acetyl choline, amylase, amyln, bilirubin, cholesterol, chorionic gonadotropin, creatine kinase (e.g., CK-MB), creatine, DNA, fructosamine, glucose, glutamine, growth hormones, hormones, ketones, lactate, measures for oxidative stress (such as 8-iso PGF2gamma), peroxide, prostate-specific antigen, prothrombin, RNA, thyroid stimulating hormone, and troponin. The concentration of drugs, such as, for example, antibiotics (e.g., gentamicin, vancomycin, and the like), biguanides, digitoxin, digoxin, drugs of abuse, GLP-1, insulin, PPAR agonists, sulfonylureas, theophylline, thiazolidinediones, and warfarin, may also be determined.

Moreover, within the scope of the present invention, the transmitter units of the analyte monitoring system 110 may be configured to directly communicate with one or more of the remote terminal 140 or the fluid delivery device 120. Furthermore, within the scope of the present invention, additional devices may be provided for communication in the analyte monitoring system 100 including additional receiver/data processing unit, remote terminals (such as a physician's terminal and/or a bedside terminal in a hospital environment, for example).

In addition, within the scope of the present invention, one or more of the analyte monitoring system 110, the fluid delivery device 120 and the remote terminal 140 may be configured to communicate over a wireless data communication link such as, but not limited to RF communication link, Bluetooth communication link, infrared communication link, or any other type of suitable wireless communication connection between two or more electronic devices, which may further be unidirectional or bi-directional communication between the two or more devices. Alternatively, the data communication link may include wired cable connection such as, for example, but not limited to RS232 connection, USB connection, or serial cable connection.

The fluid delivery device 120 may include in one embodiment, but not limited to, an external infusion device such as an external insulin infusion pump, an implantable pump, a pen-type insulin injector device, a patch pump, an inhalable infusion device for nasal insulin delivery, or any other type of suitable delivery system. In other embodiment, the fluid delivery device 120 may be configured to deliver other types of therapeutic fluids for treating different physiological conditions such as cancer. In addition, the remote terminal 140 in one embodiment may include for example, a desktop computer terminal, a data communication enabled kiosk, a laptop computer, a handheld computing device such as a personal digital assistant (PDAs), or a data communication enabled mobile telephone.

Referring back to FIG. 1, in one embodiment, the analyte monitoring system 110 includes a strip port configured to receive a test strip for capillary blood glucose testing. In one aspect, the glucose level measured using the test strip may in addition, be configured to provide periodic calibration of the analyte sensors of the analyte monitoring system 110 to assure and improve the accuracy of the analyte levels detected by the analyte sensors.

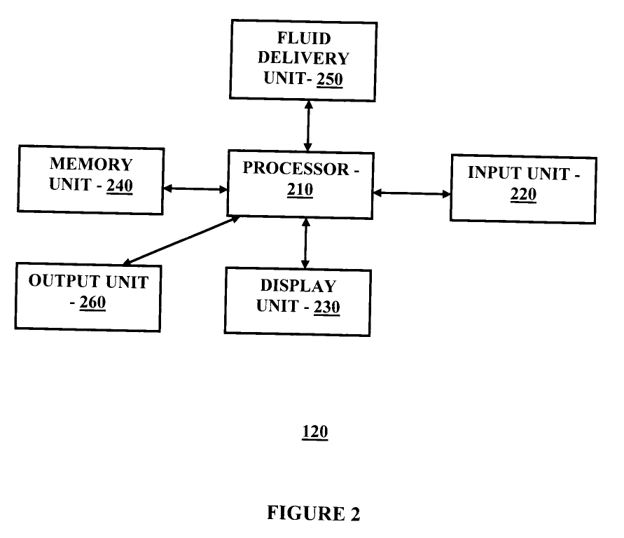

FIG. 2 is a block diagram of an fluid delivery device of FIG. 1 in one embodiment of the present invention. Referring to FIG. 2, the fluid delivery device 120 in one embodiment includes a processor 210 operatively coupled to a memory unit 240, an input unit 220, a display unit 230, an output unit 260, and a fluid delivery unit 250. In one embodiment, the processor 210 includes a microprocessor that is configured to and capable of controlling the functions of the fluid delivery device 120 by controlling and/or accessing each of the various components of the fluid delivery device 120. In one embodiment, multiple processors may be provided as safety measure and to provide redundancy in case of a single processor failure. Moreover, processing capabilities may be shared between multiple processor units within the fluid delivery device 120 such that pump functions and/or control maybe performed faster and more accurately.

Referring back to FIG. 2, the input unit 220 operatively coupled to the processor 210 may include a jog dial, a key pad buttons, a touch pad screen, or any other suitable input mechanism for providing input commands to the fluid delivery device 120. More specifically, in the embodiments that include a jog dial input device, or a touch pad screen, for example, the patient or user of the fluid delivery device 120 is able to manipulate the respective jog dial or touch pad in conjunction with the display unit 230 which performs as both a data input and output units. The display unit 230 may include a touch sensitive screen, an LCD screen, or any other types of suitable display unit for the fluid delivery device 120 that is configured to display alphanumeric data as well as pictorial information such as icons associated with one or more predefined states of the fluid delivery device 120, or graphical representation of data such as trend charts and graphs associated with the insulin infusion rates, trend data of monitored glucose levels over a period of time, or textual notification to the patients.

Referring to FIG. 2, the output unit 260 operatively coupled to the processor 210 may include an alarm system, e.g., one or more audible alarms including one or more tones and/or preprogrammed or programmable tunes or audio clips, or vibratory alert features having one or more pre-programmed or programmable vibratory alert levels. In one embodiment, the vibratory alert may also assist to notify a user to prime the infusion tubing to minimize the potential for air or other undesirable material in the infusion tubing.

Also shown in FIG. 2 is the fluid delivery unit 250 which is operatively coupled to the processor 210 and configured to deliver the insulin doses or amounts to the patient from the insulin reservoir or any other types of suitable containment for insulin to be delivered (not shown) in the fluid delivery device 120 via an infusion set coupled to a subcutaneously positioned cannula under the skin of the patient.

Referring yet again to FIG. 2, the memory unit 240 may include one or more of a random access memory (RAM), read only memory (ROM), or any other types of data storage units that is configured to store data as well as program instructions for access by the processor 210 and execution to control the fluid delivery device 120 and/or to perform data processing based on data received from the analyte monitoring system 110, the remote terminal 140, the patient 130 or any other data input source.

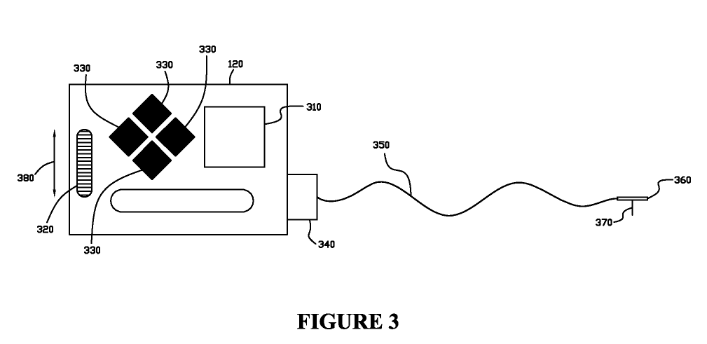

FIG. 3 is a block diagram of an fluid delivery device with a jog wheel user interface and retractable tubing in one embodiment of the present invention. Referring to FIG. 3, fluid delivery device 120 is provided with a display unit 310 for output visual display, a jog wheel user interface unit 320, and a plurality of input switches. In one embodiment, the user of the insulin delivery unit 120 may manipulate one or more of the jog wheel user input unit 320 or the plurality of switches 330 to program or control the fluid delivery device 120. The display unit 310 in one embodiment is configured to provide visual indication of the operating status as well as other information associated with the operation of the fluid delivery device 120, and may include one or more icon representations, alphanumeric representations, color or graphical representations and background light feature. In one embodiment, the display unit 310 may include an LCD display unit or any other similar display unit that may be integrated with the housing of the fluid delivery device 120.

Referring back to FIG. 3, the jog wheel user interface unit 320 may be configured to rotate about its centre by rotational movement of, for example, the user's finger on the jog wheel user interface unit 320 substantially in the direction as shown by the arrow 380. The movement of the jog wheel user interface unit 320 may be associated with one or more displayed information on the display unit 310 such that the user may manipulate or scroll through a menu structure displayed on the display unit 310 for controlling or programming the fluid delivery device 120. Additionally, within the scope of the present invention, the jog wheel user interface unit 320 may be further configured to be depressed by, for example, the user's finger such that the depression of the jog wheel user interface unit 320 may be associated with a selection function associated with a predetermined displayed item. In other words, in one embodiment, the user of the fluid delivery device 120 may manipulate the jog wheel user interface unit 320 to navigate through the menu structure displayed on the display unit 310 of the fluid delivery device 120.

Referring again to FIG. 3, each of the plurality of switches or input buttons 330 may be programmed for a predetermined function associated with the operation of the fluid delivery device 120. For example, of the plurality of input buttons 330 may be a dedicated bolus button which may be activated to initiate the delivery of a determined bolus dosage. Additionally, other frequently used or user desired functions may be programmed in the fluid delivery device 120 such that each of the plurality of input buttons 330 may be configured to perform a particular task. For example, one of the plurality of input buttons 330 may be configured to provide backlighting feature to illuminate the display unit 310 in a dark environment.

Referring yet again to FIG. 3, the fluid delivery device 120 is provided with a retractable tubing unit 340 (e.g., variable length tubing unit) mounted to the housing of the fluid delivery device 120 along the fluid path connecting the reservoir (not shown) within the housing, to an infusion tubing 350 connected to an infusion set 360 that is also provided with a cannula a portion of which is for placement under the skin of the patient for infusion of a therapeutic agent, for example, insulin. As discussed in further detail below, in one embodiment of the present invention, the retractable tubing unit 340 is configured to adjust the length of the infusion tubing 350 so that the patient or user of the fluid delivery device 120 may vary or modify the length of the infusion tubing 350 relative to the fluid delivery device 120 and the infusion site where the cannula 370 is positioned.

FIG. 4 is a detailed view of the retractable tubing unit of the infusion device of FIG. 3 in one embodiment of the present invention. A mechanism may be provided for retracting any excess extended tubing thereby preventing constriction and tangling of the tubing. The device may retract the tubing into the housing where the tubing is rewound in a manner which also prevents constriction and tangling of the tubing inside the housing. The device may also be constructed to facilitate servicing of the tubing through easy removal of the coiled tubing from the housing of the device.

Referring to FIG. 4, the retractable tubing unit 340 in one embodiment includes a core section 410 which is configured to rotate about its center portion 420 on its longitudinal axis to provide extension or shortening of the infusion tubing 350 extending from the housing of the fluid delivery device 120. More particularly, in one embodiment, the infusion tubing 350 is configured to wrap around the core section 410, e.g., along a predefined groove or indentation path provided on the outer surface of the core section 410 to guide the infusion tubing 350. Referring again to FIG. 4, the center portion 420 of the core section 410 in one embodiment is coupled to a motor or an actuation unit of the fluid delivery device 120 (or alternatively provided in the retractable tubing unit 340) such that the user may, by operation of a control function through one or more of the user interface devices (such as the jog wheel user interface unit 320 or one or more of the plurality of buttons 33), and under the control of the processor 210 (FIG. 2) rotate the core section 410 about its center portion 420 which in turn, extends or retracts the infusion tubing 350 segment outside of the retractable tubing unit 340.

In one embodiment, the rotation of the core section 410 may be implemented at a relatively slow rate such that potential kinking in the infusion tubing within the retractable tubing unit 340 is avoided. Additionally, the paced, relatively slow rate at which the rotation of the core section 410 is implemented may provide additional protection from accidental withdrawal or dislocation of the cannula 370.

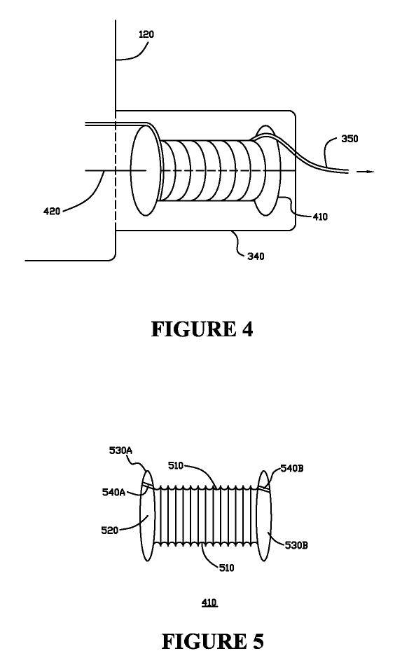

In this manner, in one embodiment of the present invention, the user may modify or vary the length of the infusion tubing which is connecting the infusion device 120 to the infusion set 360 FIG. 5 is a detailed view of the retractable infusion tubing core segment of the retractable infusion tubing section of FIG. 4 in one embodiment of the present invention. Referring to FIG. 5, the core section 410 of the retractable tubing unit 340 in one embodiment includes the plurality of grooves or indentations 510 which are configured to guide the infusion tubing 350 during the rotational movement of the core section 410 such that the infusion tubing may be properly guided in particular, during the tuning retraction process. In this manner, potential overlapping of the tubing 350 within the retractable tubing unit 340 may be avoided, and additionally the potential for occlusion in the fluid path of the tubing 350 due to, for example, kinking in the tubing 350 may be avoided.

Referring again to FIG. 5, the core section 410 in one embodiment may include end sections 530A, 530B, each provided with a guide groove 540A, 540B, respectively. the guide grooves 540A, 540B are configured to properly seat or guide the tubing 350 on the core section 410 to minimize potential occlusion or kinking in the tubing 350. Furthermore, as shown in FIG. 5, the core section 410 is provided with a bore 520 which is configured to receive a spindle shaft or a similar device that may be coupled to the motor or the actuation unit operatively coupled to the processor 210 of the fluid delivery device 120 for executing the rotational movement of the core section 410.

In addition, while the retractable tubing unit 340 is provided on the housing of the fluid delivery device 120, within the scope of the present invention, the retractable tuning unit 340 may be provided at any suitable location along the fluid path guided by the tubing 350 between the infusion set 360 and the housing of the fluid delivery device 120. Furthermore, within the scope of the present invention, the rotatable mechanism for providing the retraction and extension of the infusion tubing 350 may be implemented in accordance with other suitable approaches that substantially minimize potential occlusion in the tubing 350, and further, which may be controlled by the user of the fluid delivery device 120.

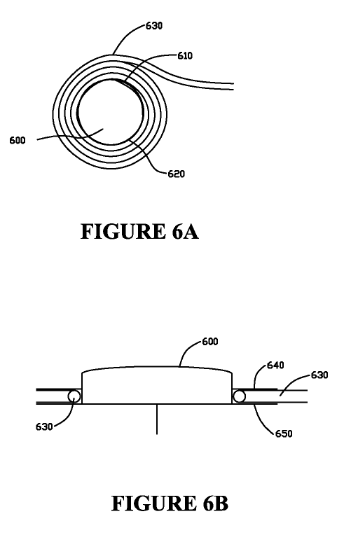

FIGS. 6A and 6B are top planar view and side cross-sectional view, respectively, of the retractable infusion tubing unit in accordance with another embodiment of the present invention. Referring to FIG. 6A, in one embodiment, there is provided a rotatable retracting mechanism 620 coupled to an infusion set tubing 630, and substantially around which, the infusion set tubing 630 is provided. In particular, a tubing attachment segment 610 is provided and configured to attach the infusion set tubing 630 to the rotatable retracting mechanism 620.

Referring back to FIG. 6A, it can be seen that the tubing attachment segment 610 is also configured to couple to the internal housing of the infusion device so as to establish a fluid path from the reservoir of the infusion device to cannula at the infusion site. Referring now to FIG. 6B, in one embodiment, an upper layer 640 and a lower layer 650 may be provided to substantially retain the infusion set tubing 630 in the desired or predetermined position relative to the rotatable retracting mechanism 620. Furthermore, the upper layer 640 may be configured to retain the infusion set tubing 630 substantially flat and untwisted.

In addition, in the case of the infusion device including an on-body micro-pump such as a patch pump, wherein the infusion tubing rotatable retracting mechanism 620 is provided on the on-body micro-pump, the lower layer 650 may be configured to provide a barrier between the patient's skin and the external surface of the infusion set tubing so as to minimize potential skin irritation where the infusion set tubing may be in contact with the patient's skin if the lower layer 650 is not provided.

In this manner, in accordance with the various embodiments of the present invention, there are provided methods and system for an intuitive and robust user interface/input mechanism for controlling and/or programming a remote controller for an infusion device, an analyte monitoring system receiver unit, or as part of the infusion device interface unit. In addition, within the scope of the present invention, there are provided methods and apparatus for retractable infusion set tubing length to provide comfort and added ease of wear for patients or users of the infusion devices.

An analyte monitoring device in one embodiment of the present invention includes a housing, a processor disposed in the housing for performing data processing, and a rotatable user interface device coupled to the housing, the rotatable user interface device operatively coupled to the processor for providing user input commands.

The rotatable user interface device may include a jog wheel.

Also, the rotatable user interface device may be further configured for activation upon depression.

There may also be provided a display unit coupled to the housing, the display unit operatively coupled to the processor to display one or more output signals responsive to a respective one or more of the user input commands.

In addition, there may be provided an infusion device, the infusion device including a retractable tubing section provided along a fluid path from the housing, where the retractable tubing section may be coupled to the infusion device housing.

In a further embodiment, the retractable tubing section may be provided on the infusion device housing and configured to house a portion of an infusion tubing connectable to the infusion device housing.

The retractable tubing section may include a core section configured to wind the portion of the infusion tubing substantially around the outer surface of the core section, and where the core section may be provided with a groove defining a concentric path on the outer surface of the core section.

In one aspect, the groove may be configured to receive the portion of the infusion tubing such that the portion of the infusion tubing is substantially disposed on the outer surface of the core section along the groove.

An infusion device in accordance with another embodiment of the present invention includes a housing, a processor disposed in the housing for performing data processing, a user interface unit disposed on the housing and operatively coupled to the processor, a display unit disposed on the housing and operatively coupled to the processor, and a retractable tubing section provided along a fluid path from the housing.

In one aspect, the retractable tubing section may be provided on the housing and configured to house a portion of an infusion tubing connectable to the housing.

The retractable tubing section may include a core section configured to wind the portion of the infusion tubing substantially around the outer surface of the core section.

The core section may be provided with a groove defining a concentric path on the outer surface of the core section.

The groove may be configured to receive the portion of the infusion tubing such that the portion of the infusion tubing is substantially disposed on the outer surface of the core section along the groove.

There may also be provided an infusion set coupled to the retractable tubing section.

A method in accordance with still another embodiment includes providing a tubing retraction and extension mechanism along a fluid path from a housing of an infusion device, and adjusting a length of an infusion tubing by manipulating the tubing retraction and extension mechanism to modify the length of the infusion tubing between a cannula and the infusion device.

In one aspect, adjusting may include extending the length of the infusion tubing between the cannula and the infusion device.

In still another aspect, adjusting may include shortening the length of the infusion tubing between the cannula and the infusion device.

In a further aspect, providing may include coupling the tubing retraction and extension mechanism to the housing of the infusion device.

Moreover, the method may also include positioning a cannula under a skin of a patient, wherein the cannula is in fluid communication with the infusion tubing, where the cannula may be connected to the infusion tubing to provide a continuous fluid path from the infusion device to an infusion site of the patient.

In one aspect, the infusion device may include an insulin pump.

An on-body micropump in accordance with still another embodiment includes a housing including a reservoir, a cannula having at least a portion transcutaneously positioned under the skin of a patient, and a retractable infusion tubing coupled to the reservoir and the cannula to maintain a fluid path between the reservoir and the cannula.

Various other modifications and alterations in the structure and method of operation of this invention will be apparent to those skilled in the art without departing from the scope and spirit of the invention. Although the invention has been described in connection with specific preferred embodiments, it should be understood that the invention as claimed should not be unduly limited to such specific embodiments. It is intended that the following claims define the scope of the present invention and that structures and methods within the scope of these claims and their equivalents be covered thereby.

What is claimed is:

1-10. (canceled)

11. An infusion device, comprising:
a housing;
a processor disposed in the housing for performing data processing;
a user interface unit disposed on the housing and operatively coupled to the processor;
a display unit disposed on the housing and operatively coupled to the processor; and
a retractable tubing section provided along a fluid path from the housing;
wherein the processor is configured to control the operation of the retractable tubing section based at least in part, on one or more commands received from the user interface unit.

12. The device of claim 11 wherein the retractable tubing section is provided on the housing and configured to house a portion of an infusion tubing connectable to the housing, wherein variation of the infusion tubing length extending from the retractable tubing section is controlled by the processor.

13. The device of claim 12 wherein the retractable tubing section includes a core section configured to wind the portion of the infusion tubing substantially around the outer surface of the core section.

14. The device of claim 13 wherein the core section is provided with a groove defining a concentric path on the outer surface of the core section.

15. The device of claim 14 wherein the groove is configured to receive the portion of the infusion tubing such that the portion of the infusion tubing is substantially disposed on the outer surface of the core section along the groove.

16. The device of claim 11 further including an infusion set coupled to the retractable tubing section.

17-24. (canceled)

25. The device of claim 11 wherein the one or more commands received from the user interface unit controls the rate of variation of an infusion tubing length extending out of the retractable tubing section.

26. The device of claim 25 wherein the retractable tubing section is provided on the housing and configured to house a portion of an infusion tubing connectable to the housing, the retractable tubing section including a core section coupled to the processor.

27. The device of claim 26 wherein the processor is configured to control the operation of the core section to vary the length of the infusion tubing extending out of the housing.

28. The device of claim 27 wherein the processor controls the core section rotate about its center axis at one or more predetermined speeds for a preset time period.

29. The device of claim 28 wherein the one or more predetermined speeds is defined by a control signal received from the user interface.

30. The device of claim 28 wherein the preset time period is defined by a control signal received from the user interface.

31. The device of claim 11 wherein the infusion device includes an insulin pump.

32. An infusion pump, comprising:
a housing;
a reservoir coupled to the housing, the reservoir containing a fluid;
a processor disposed in the housing for performing data processing;
a user input unit operatively coupled to the processor;
an output unit operatively coupled to the processor; and
a retractable tubing section coupled to the processor in the housing, the retractable tubing section including an infusion tubing for delivering the fluid from the reservoir in the housing;
wherein the processor is configured to control one or more of delivery of the fluid from the housing, or varying the length of the infusion tubing.
33. The apparatus of claim 32 wherein the processor is configured to control the delivery of the fluid or vary the length of the infusion tubing based on one or more control signals received from the user input unit.

34. The apparatus of claim 32 wherein the output unit includes a display unit to display an output associated with one or more of the fluid delivery from the reservoir, or the infusion tubing length.

35. The apparatus of claim 32 wherein the output displayed on the output unit includes one or more of a graphical output, a text output, an audible output, or a vibratory output.

36. The apparatus of claim 32 wherein the fluid includes insulin.

37. The apparatus of claim 32 wherein the processor in configured to generate one or more alarm signals based on one or more conditions associated with the fluid delivery or the tubing length variation.

38. The apparatus of claim 37 wherein, under the control of the processor, the one or more alarm signals is output to the output unit.

39. A method of providing fluid delivery, comprising:
coupling a user interface unit to a housing;
coupling a display unit to the housing;
providing a retractable tubing section coupled to the housing;
operatively coupling a processor disposed in the housing to the user interface unit, the display unit and the retractable tubing section; and
controlling the operation of the retractable tubing section based at least in part, on one or more commands received from the user interface unit.

40. The method of claim 39 including:
connecting an infusion tubing to the retractable tubing section; and
varying the length of the infusion tubing section in response to one or more signals from the processor.

41. The method of claim 40 including connecting an infusion set to the infusion tubing.

42. The method of claim 40 including:
receiving one or more commands from the user interface unit; and
controlling the infusion tubing length extending out of the retractable tubing section in response to the one or more commands.

43. The method of claim 39 including coupling a reservoir to the housing.

44. The method of claim 43 including dispensing a fluid from the reservoir under the control of the processor.

45. The method of claim 44 wherein the fluid is insulin.

46. A method, comprising:
providing a housing;
coupling a reservoir to the housing, the reservoir containing a fluid;
disposing a processor in the housing to perform data processing;
operatively coupling a user interface unit to the processor;
coupling a retractable tubing section including an infusion tubing to the housing;
establishing fluid contact between the infusion tubing and the reservoir; and
configuring the processor to control one or more of delivery of the fluid from the housing, or varying the length of the infusion tubing.

47. The method of claim 46 including:
receiving a control signal from the user input unit; and
executing a command associated with the control signal to control the delivery of the fluid or vary the length of the infusion tubing.

48. The method of claim 46 outputting an output signal associated with one or more of the fluid delivery from the reservoir, or the infusion tubing length.

49. The method of claim 48 wherein the output signal includes one or more of a graphical output, a text output, an audible output, or a vibratory output.

50. The method of claim 46 wherein the fluid includes insulin.

51. The method of claim 46 including generating one or more alarm signals based on one or more conditions associated with the fluid delivery or the tubing length variation.
